(19) United States
(12) Patent Application Publication
(10) Pub. No.: US 2024/0053314 A1
(43) Pub. Date: Feb. 15, 2024

(54) PURGE GAS MONITOR

(71) Applicant: Preddio Technologies Inc., North Andover, MA (US)

(72) Inventors: Aaron T. Ganick, Boxford, MA (US); Maxwell Nicolaus Little, Newton, MA (US); Joseph Robert Stauff, Westport, CT (US)

(21) Appl. No.: 18/449,229

(22) Filed: Aug. 14, 2023

Related U.S. Application Data
(60) Provisional application No. 63/371,476, filed on Aug. 15, 2022.

(51) Int. Cl. G01N 33/00; G01D 11/24; H05K 1/00

(52) U.S. Cl. CPC G01N 33/0036; G01N 33/0063; G01D 11/245; H05K 1/00; H05K 2201/10151

(57) ABSTRACT

A purge gas monitoring device and system are provided. The purge gas monitoring system includes a brew tank, a purge gas monitor coupled to a pipe connected to the brew tank, and a controller electronically coupled to the purge gas monitor. The controller is configured to: receive a plurality of oxygen concentration measurements from the purge gas monitor over a period of time, fit the plurality of oxygen concentration measurements onto a curve, and extrapolate the curve to estimate a time when oxygen concentration will reach a threshold.

### CROSS-REFERENCE TO RELATED APPLICATION

This application claims the benefit of the filing date of U.S. Provisional Application Ser. No. 63/371,476, filed Aug. 15, 2022, entitled, “Purge Gas Monitor”, which is hereby incorporated by reference as if fully set forth herein.

### TECHNICAL FIELD

This disclosure generally relates to a gas monitoring device. More specifically, this disclosure relates to a purge gas monitor that may be used in brewing systems.

### BACKGROUND

In the brewing industry, preventing the inclusion of oxygen, which causes alcoholic beverages to go bad, is an important necessity. At the beginning of the brewing process, brewing tanks, which have been open to the air, are purged of oxygen using a filler gas such as carbon dioxide (CO₂) or nitrogen (N₂).

Currently, breweries typically determine that a tank has been fully purged using prohibitively expensive oxygen sensors or a manual “smell test” that may be hazardous to workers. These tests must be performed by hand, resulting in inefficiencies and loss of an expensive commodity when the flow of purge gas is left on for longer than necessary.

Moreover, although it may be convenient to run purge sequences overnight during the brewery's off-hours, without an automatic test or a way to control the flow of purge gas, running purge sequences at night would waste a large amount of gas and be prohibitively expensive.

In addition, oxygen sensors capable of measuring concentrations of oxygen present at the end of a purge process, which are around 15 parts per billion (ppb), are not affordable and often have service lifetimes of less than two years.

Therefore, there is a need for a cheaper and more efficient device and method of automatically detecting when a tank has been fully purged and taking actions in response thereto.

Moreover, current systems that monitor a purge process typically use oxygen sensors that measure a percentage or partial pressure of oxygen in the air without regard that only some of gaseous oxygen may saturate and become dissolved in equilibrium with a fluid.

Therefore, there is also a need to determine an amount of oxygen that can be dissolved into a brew of a brewing system (such as beer) to determine an impact of gaseous oxygen on the brew.

### BRIEF SUMMARY

A first aspect of this disclosure pertains to a purge gas monitor including a housing assembly coupled to a base assembly, and a sensor assembly provided in the base assembly, wherein the sensor assembly includes an oxygen sensor.

A second aspect of this disclosure pertains to the purge gas monitor of the first aspect, wherein the housing assembly further includes a lens coupled to a top end of a housing, wherein a bottom end of the housing is configured to be coupled to the base assembly.

A third aspect of this disclosure pertains to the purge gas monitor of the first aspect, wherein sensor assembly further includes a sensor printed circuit board (PCB) coupled to the oxygen sensor.

A fourth aspect of this disclosure pertains to the purge gas monitor of the third aspect, wherein the sensor assembly is coupled to base assembly through a gasket provided between a bottom surface of the oxygen sensor and the base assembly.

A fifth aspect of this disclosure pertains to the purge gas monitor of the fourth aspect further including a vent provided between the bottom surface of the oxygen sensor and the gasket.

A sixth aspect of this disclosure pertains to the purge gas monitor of the first aspect further including a battery assembly, wherein the battery assembly is provided above the sensor assembly and is enclosed by the housing assembly when the purge gas monitor is assembled.

A seventh aspect of this disclosure pertains to the purge gas monitor of the sixth aspect, wherein the battery assembly further including a carrier for receiving a batter therein; a first printed circuit board (PCB) provided on a first exterior surface of the carrier; and a second PCB is provided on a second exterior surface of the carrier.

An eighth aspect of this disclosure pertains to the purge gas monitor of the seventh further including a shroud attachable to the carrier, wherein the shroud encloses the first PCB when the shroud is attached to the carrier; and a battery retainer attachable to the carrier, wherein the battery retainer retains the battery within the carrier when the battery retainer is attached to the carrier.

A ninth aspect of this disclosure pertains to the purge gas monitor of the eighth aspect, wherein the shroud and the battery retainer are provided on opposite sides of the carrier.

A tenth aspect of this disclosure pertains to the purge gas monitor of the first aspect, wherein a seal is provided in between the housing assembly and the base assembly.

An eleventh aspect of this disclosure pertains to a brewing system including a brew tank, a purge gas monitor coupled to a pipe connected to the brew tank; and a controller electronically coupled to the purge gas monitor, wherein the controller is configured to receive a plurality of oxygen concentration measurements from the purge gas monitor over a period of time, fit the plurality of oxygen concentration measurements onto a curve, and extrapolate the curve to estimate a time when oxygen concentration will reach a threshold.

A twelfth aspect of this disclosure pertains to the brewing system of the eleventh aspect further including a valve coupled to an actuator, wherein the controller is further configured to in response to reaching the estimated time, control the actuator to actuate the valve.

A thirteenth aspect of this disclosure pertains to the brewing system of the twelfth aspect, wherein the valve controls an infill gas flow into the brew tank.

A fourteenth aspect of this disclosure pertains to the brewing system of the eleventh aspect, wherein the pipe that the purge gas monitor coupled to is an outlet pipe from the brew tank.

A fifteenth aspect of this disclosure pertains to the brewing system of the eleventh aspect, wherein the pipe that the purge gas monitor coupled to is an inlet pipe to the brew tank.

A sixteenth aspect of this disclosure pertains to the brewing system of the eleventh aspect, wherein the pipe further includes a first section coupled to a second section, wherein the purge gas monitor is coupled to the second section of the pipe, and wherein a first end of the first section of the pipe is coupled to an outlet pipe from the brew tank and a second end of the first section serves as an outlet of a gas from the brew tank.

A seventeenth aspect of this disclosure pertains to the brewing system of the sixteenth aspect, wherein the first section of the pipe further includes a straight portion coupled to a first funnel portion, wherein the straight portion includes a generally uniform first diameter through a length of the straight portion.

An eighteenth aspect of this disclosure pertains to the brewing system of the sixteenth aspect, wherein the first funnel portion includes a first end having the first diameter and a second end having a second diameter different from the first diameter.

A nineteenth aspect of this disclosure pertains to the brewing system of the sixteenth aspect, wherein the second section of the pipe intersects the first section of the pipe at the straight portion.

A twentieth aspect of this disclosure pertains to the brewing system of the nineteenth aspect, wherein a first end of the second section where the purge gas monitor is coupled to is substantially parallel to the straight portion and a second end of the second section where the second section intersects the first section is substantially perpendicular.

### BRIEF DESCRIPTION OF THE DRAWINGS

FIG. 1 illustrates a system diagram of a brewing system according to an embodiment.

FIG. 2 illustrates a process of determining oxygen concentration according to an embodiment.

FIG. 3 illustrates a line chart showing a relationship between oxygen concentration and time elapsed in an ideally mixed brewing tank.

FIG. 4 illustrates a perspective view of a purge gas monitor according to an embodiment.

FIG. 5 illustrates an exploded view of the purge gas monitor of FIG. 4.

FIG. 6 illustrates an exploded view of a housing assembly of the purge gas monitor of FIG. 4.

FIG. 7 illustrates a perspective view of the housing assembly of FIG. 6.

FIG. 8 illustrates an exploded view of a carrier of the purge gas monitor of FIG. 4.

FIG. 9 illustrates a perspective view of the carrier of FIG. 8.

FIG. 10 illustrates an exploded view of a battery assembly of the purge gas monitor of FIG. 4.

FIG. 11 illustrates a perspective view of the battery assembly of FIG. 10.

FIG. 12 illustrates an exploded view of a base assembly of the purge gas monitor of FIG. 4.

FIG. 13 illustrates a perspective view of the base assembly of FIG. 12.

FIG. 14 illustrates an exploded view of the base assembly of FIG. 13 coupled with the battery assembly of FIG. 11.

FIG. 15 illustrates a perspective view of the base assembly of FIG. 13 coupled with the battery assembly of FIG. 11.

FIG. 16 illustrates a side view of a purge gas monitor coupled to a pipe according to an embodiment.

FIG. 17 illustrates a side view of the pipe of FIG. 16.

FIG. 18 illustrates a top view of the pipe of FIG. 16.

FIG. 19 illustrates a bottom view of the pipe of FIG. 16.

FIG. 20 illustrates an example configuration of monitoring an oxygen purge using the purge gas monitor and the pipe of FIG. 16.

FIG. 21 illustrates an example configuration of monitoring a carbon dioxide purge using the purge gas monitor of FIG. 4.

FIG. 22 illustrates a side view of a chill monitor according to an embodiment.

FIG. 23 illustrates a side view of a temperature monitor according to an embodiment.

FIG. 24 illustrates a side view of a rinse monitor according to an embodiment.

FIG. 25 illustrates a side view of a pH monitor according to an embodiment.

FIG. 26 illustrates a side view of a dissolved oxygen (DO) monitor according to an embodiment.

FIG. 27 illustrates a side view of a steam monitor according to an embodiment.

Before explaining the disclosed embodiment of this disclosure in detail, it is to be understood that the invention is not limited in its application to the details of the particular arrangement shown, since the invention is capable of other embodiments. Exemplary embodiments are illustrated in referenced figures of the drawings. It is intended that the embodiments and figures disclosed herein are to be considered illustrative rather than limiting. Also, the terminology used herein is for the purpose of description and not of limitation.

### DETAILED DESCRIPTION

While this invention is susceptible to embodiments in many different forms, there are shown in the drawings and will be described in detail herein specific embodiments with the understanding that the present disclosure is an exemplification of the principles of the invention. It is not intended to limit the invention to the specific illustrated embodiments. The features of the invention disclosed herein in the description, drawings, and claims can be significant, both individually and in any desired combinations, for the operation of the invention in its various embodiments. Features from one embodiment can be used in other embodiments of the invention.

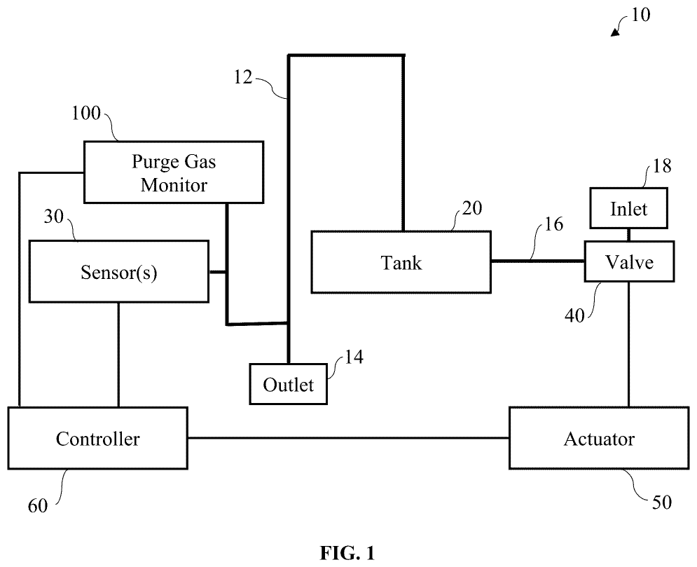

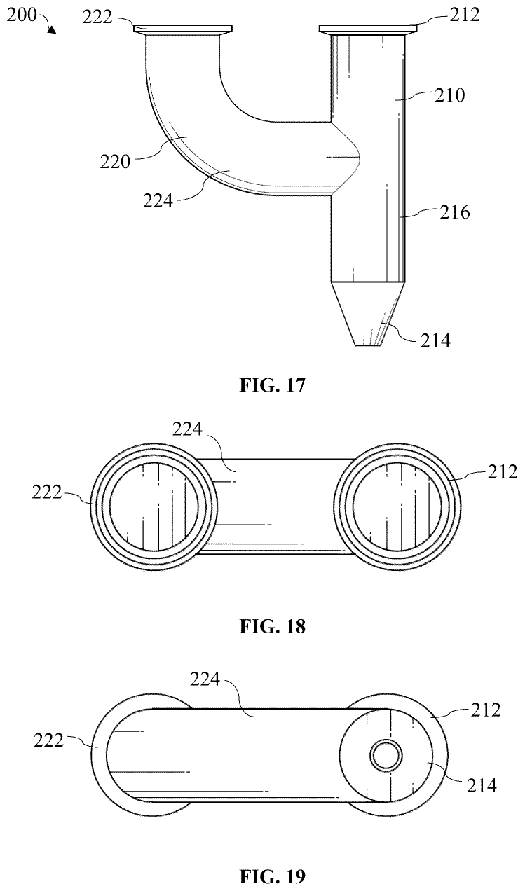

As shown in FIGS. 1-19, embodiments of this disclosure include a purge gas monitor. Referring to FIG. 1, a brewing system 10 according to some embodiments may include a purge gas monitor 100 coupled to an outlet pipe 12. The outlet pipe 12 may serve as a conduit for allowing purge gas to escape from a tank 20 such as a brew tank. The outlet pipe 12 may include an outlet 14 for allowing the purge gas to escape into the atmosphere or other components. The purge gas may be oxygen, carbon dioxide, or other gas in need of monitoring depending on the specific implementation.

The outlet pipe 12 may further be coupled to one or more sensors 30. The sensor 30 may be a temperature sensor, a humidity sensor, a pressure sensor, and/or other sensors that may be deployed in the brewing system 10.

The tank 20 may further be coupled to an inlet pipe 16. The inlet pipe 16 may serve as a conduit for injecting filler gas into the tank 20. The inlet pipe 16 may include an inlet 18 for receiving filler gas from an external component. The filler gas may be carbon dioxide, nitrogen, or other gas known in the art.

A valve 40 may be provided to the inlet pipe 16 such that the valve 40 can be actuated to control a flow of the filler gas from the inlet 18 into the tank 20. An actuator 50 may be coupled to the valve 40 for actuating the valve 40.

The purge gas monitor 100, the sensor 30, and/or the actuator 50 may be electronically connected to a controller 60. Such connections may be through hardwire and/or through wireless connectivity such as Bluetooth, LoRa, Zigbee, WiFi, or the likes, or through a combination of wired and wireless connections thereof.

The controller 60 may be configured to receive data from the purge gas monitor 100 and/or the sensor 30, and based on the data received, the controller 60 may further be configured to control the actuator 50 to actuate the valve 40.

In practice, the purge gas monitor 100 may be configured to monitor a gas leaving through the outlet pipe 12 from the tank 20 during purging. The monitoring of the gas can be continuously, near continuously, periodically, randomly, and/or at some other time intervals.

To determine when purging is complete, the controller 60 may extrapolate data from when oxygen levels were resolvable (at the start of the purge) by fitting data to a mathematical model of the purge process. The sensor 30 may also monitor humidity, temperature, and other useful metrics for reporting.

The controller 60 may receive data from the purge gas monitor 100 and/or the sensor 30. The data received by the controller 60 may be displayed through one or more displays, reported to external devices, and/or saved to a memory (such as locally to a hard drive or remotely to a server).

When the purge gas is well mixed with remaining air inside the tank 20 before exiting, the control 60 may determine when the oxygen has reached an acceptable threshold by process 61 as shown in FIG. 2.

At step 62, take a plurality of measurements of oxygen concentrations, via the purge gas monitor 100, over a period of time (such as at t₁, t₂, t₃, . . . tₙ), during an initial stage of a purge, when oxygen levels are well within monitoring range of the purge gas monitor 100.

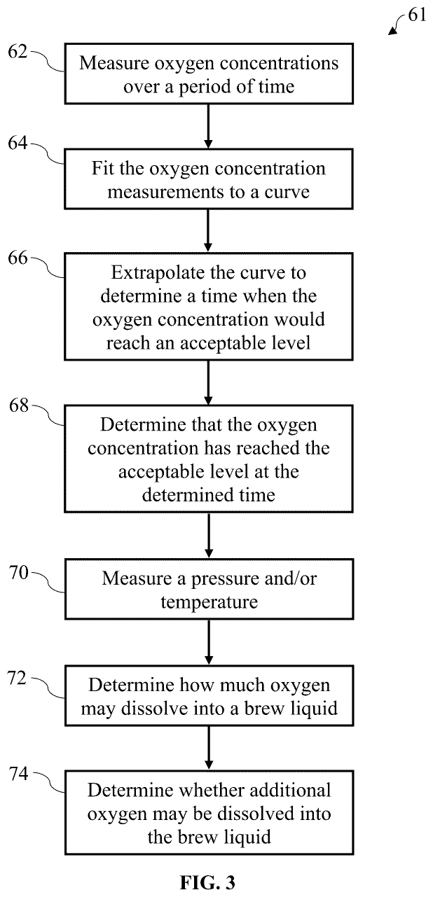

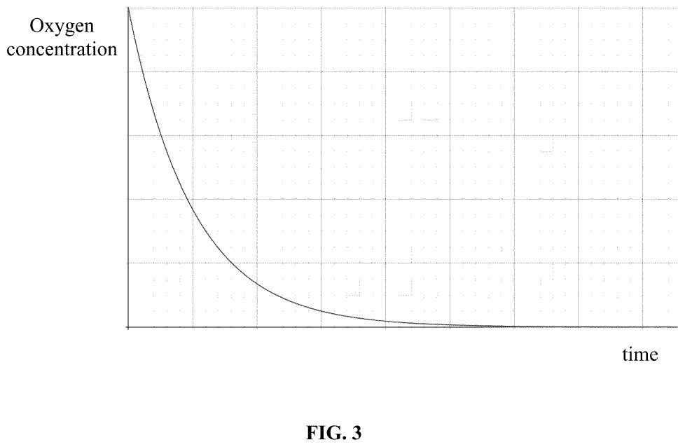

At step 64, the controller 60 may fit the measurements to a curve represented by equation y=Ae^{-ct} as shown in FIG. 3 using techniques such as least squares regression.

At step 66, the controller 60 may extrapolate the curve and determine at what time would the oxygen level reaches an acceptable level (such as about 100 ppb, about 75 ppb, about 50 ppb, about 25 ppb, about 15 ppb, about 10 ppb, about 5 ppb, about 1 ppb, or less than about 1 ppb). For example, based on the plurality of measurements, the controller 60 may determine that the oxygen level would reach about 15 ppb at tₙ₊ₓ.

At step 68, at tₙ₊ₓ, the controller 60 may determine that the oxygen levels are acceptably low. When the control 60 determines that the oxygen levels are acceptably low, the controller 60 may be configured to send a signal to the actuator 50 that may actuate the valve 40 to turn off a flow of filler gas. In some embodiments, the controller 60 may be configured to activity one or more visual and/or audio indicators such as flashing a light or sounding a buzzer that may alert a worker to manually shut off the valve 40.

At step 70, if the sensors 30 is provided within the brewing system 10, and the sensors 30 include a thermometer and/or a barometer, the controller 60 may receive a barometric/pressure measurement and/or a temperature measure from the respective sensor 30. Alternatively, the controller 60 may assume standard atmospheric temperature and pressure (such as about 20 degree Celsius and about 1 atm) for step 72.

At step 72, the controller 60 may use the barometric and temperature measurements or assumptions from step 70 to determine how much oxygen can be dissolved into a brewing liquid (such as beer). A technique for determine dissolved gas in the brew liquid is through Henry's law represented by equation C=kP.

At step 74, the controller 60 may determine whether additional oxygen may be dissolved into the brew liquid using the oxygen concentration measured at step 62 and/or the oxygen concentration determined at step 68 against the oxygen dissolved concentration value determined at step 72. For example, if the controller 60 determines that there is about 10 ppb of gaseous oxygen, but there is already about 15 ppb of dissolved oxygen in the brewing liquid, the controller 60 may determine at step 72 that no additional gaseous oxygen may dissolve into the brewing liquid. Therefrom, the controller 60 may notify an user regarding the determination, such as no additional gaseous oxygen may dissolve into the brewing liquid, or additional gaseous oxygen may dissolve into the brewing liquid, or approximately an X amount of gaseous oxygen may dissolve into the brewing liquid.

Referring to FIGS. 4-15, the purge gas monitor 100 is illustrated in more details according to an example embodiment. The purge gas monitor 100 may include a housing assembly 110 and a base assembly 120.

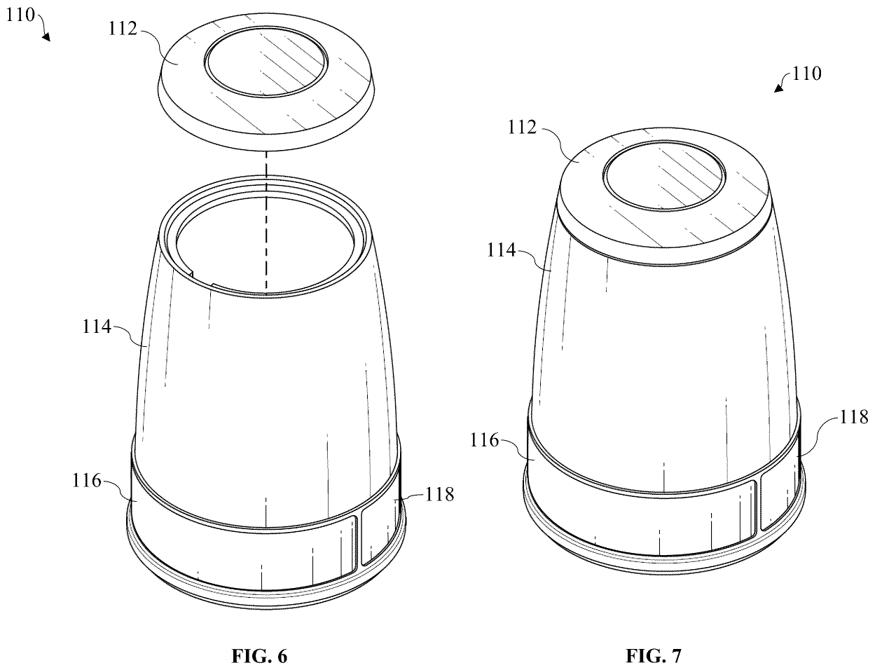

Referring to FIGS. 6 and 7 specifically, the housing assembly 110 may include a lens 112 coupled to a housing 114. The lens 112 may be sonic welded to the housing 114, though other methods of coupling the lens 112 to the housing 114 are also possible and contemplated herein. A first label 116 and a second label 118 may be affixed onto the housing 114 using suitable methods such as glue. The first label 116 and/or the second label 118 may each be provided in the form of a sticker to provide branding and/or product information pertaining to the purge gas monitor 100.

In some embodiments, the lens 112 may be provided at a top end of the housing 114, whereas the first label 116 and/or the second label 118 may each be provided on an exterior surface of the housing 114 proximal to a bottom end of the housing 114 as shown in FIG. 7.

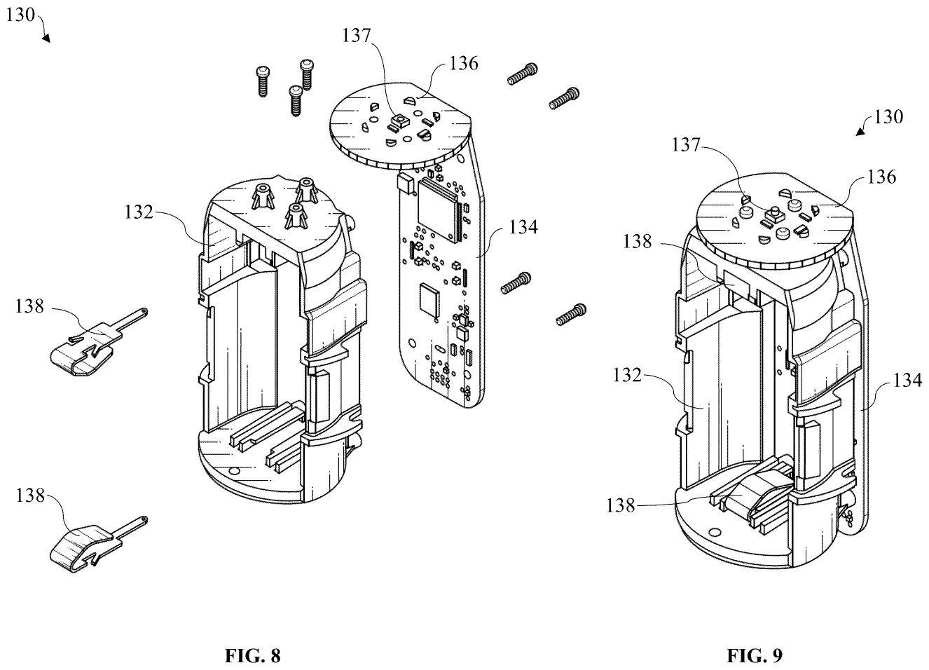

The housing 114 may be frustoconical and may enclose a carrier 130. Referring to FIGS. 8 and 9, the carrier 130 may include a battery housing 132 for receiving a battery. A first printed circuit board (PCB) 134 and a second PCB 136 may be attached to exteriors of the battery housing 132. For example, the first PCB 134 may be affixed to a rear exterior surface of the battery housing 132, and the second PCB 136 may be affixed to a top exterior surface of the battery housing 132. The first PCB 134 and the second PCB 136 may each be affixed to the battery housing 132 through one or more fasteners such as screws, though other methods of attachments are also contemplated.

The battery housing 132 may also include one or more battery contacts 138 for provided electrical power from the battery to the first PCB 134 and/or the second PCB 136. In an example embodiment, a pair of battery contacts 138 may each be provided at an opposite interior surface (such as a top interior surface and a bottom interior surface) of the battery housing 132. The battery contacts 138 may each be a leaf spring that may be soldered or otherwise coupled to the first PCB 134 and/or the second PCB 136.

The second PCB 136 may include one or more visual and/or audio indicators 137 such as light-emitting diodes (LEDs) and/or buzzers to indicate various status of the purge gas monitor 100. A portion of the lens 112 may be transparent or translucent such that the second PCB 136 may be visible through the lens 112.

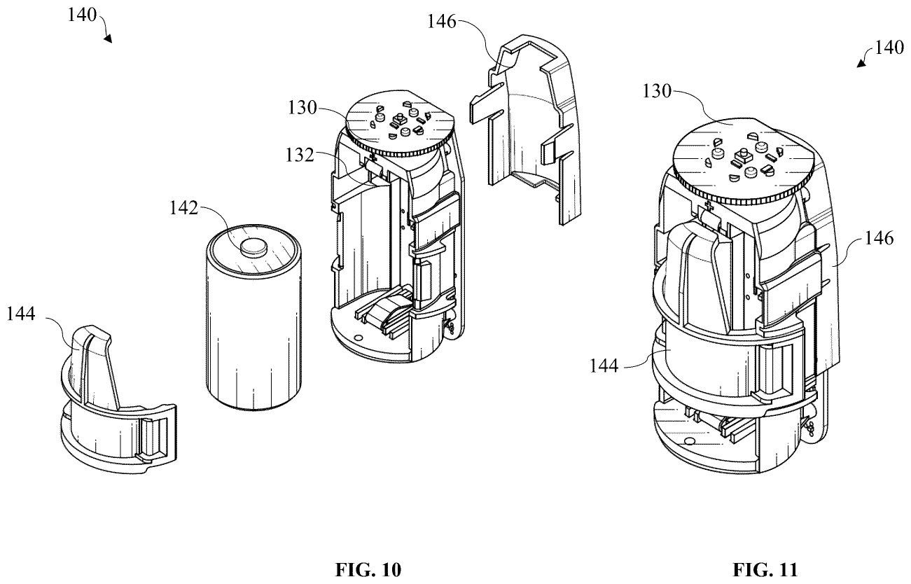

Referring to FIGS. 10 and 11, a battery assembly 140 may include the carrier 130, where the battery housing 132 of the carrier 130 may receive a battery 142 therein. When received in the battery housing 132, the positive terminal and the negative terminal of the battery 142 may each abut a battery contact 138.

Once the battery 142 is inserted into the carrier 130, a battery retainer 144 may be provided to retain the battery 142 within the carrier 130. The battery retainer 144 may include corresponding notches or groove or other mating mechanisms such that the battery retainer 144 may be mated to the battery housing 132 of the carrier 130 to retain the battery 142 therein.

A shroud 146 may be provided to cover the first PCB 134 in part or in whole. Similar to the battery retainer 144, the shroud 146 may also include corresponding notches or groove or other mating mechanisms such that the shroud 146 may be mated to the battery housing 132 of the carrier 130 to retain the first PCB 146 therein.

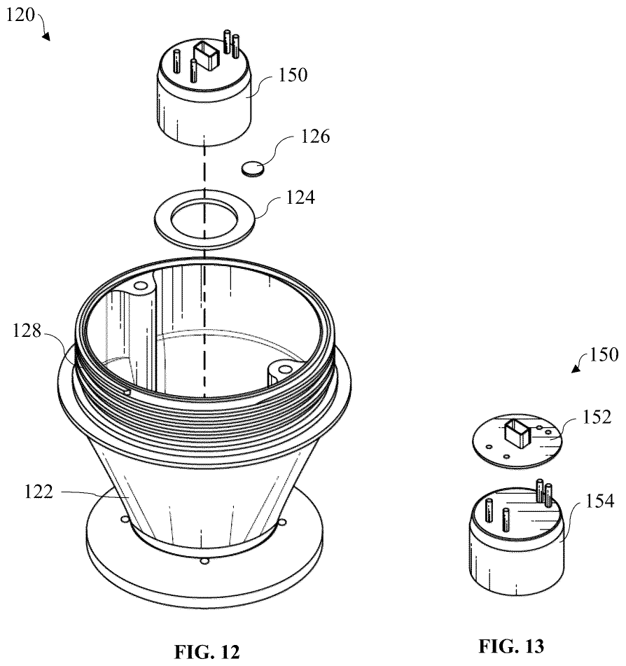

Referring to FIGS. 12 and 13, the base assembly 120 may include a base housing 122 with a sensor assembly 150 provided therein. The sensor assembly 150 may include a sensor PCB 152 coupled to a sensor 154 which may be an oxygen sensor.

The sensor assembly 150 may be coupled to a gasket 124 which may be affixed onto the base housing 122. In some embodiments, adhesive may be provided on both sides of the gasket 124 to couple the sensor assembly 150 to the base housing 122. One or more vents 126 may be provided between the gasket 124 and the sensor assembly 150. In some embodiments, the vents 124 may each be a GORE® protective vent such as the VE80205 adhesive vent.

Referring to FIGS. 14 and 15, once the sensor assembly 150 is provided inside the base housing 122 of the base assembly 120, the battery assembly 140 may be provided above the sensor assembly 150 onto the base assembly 120. Thus, when assembled, the sensor assembly 150 may be positioned proximal to a bottom of the base assembly 120 with the battery assembly 140 provided thereon. The battery assembly 140 may be attached onto the base assembly 120 through one or more fasteners such as screws, though other fastening mechanisms are also contemplated herein.

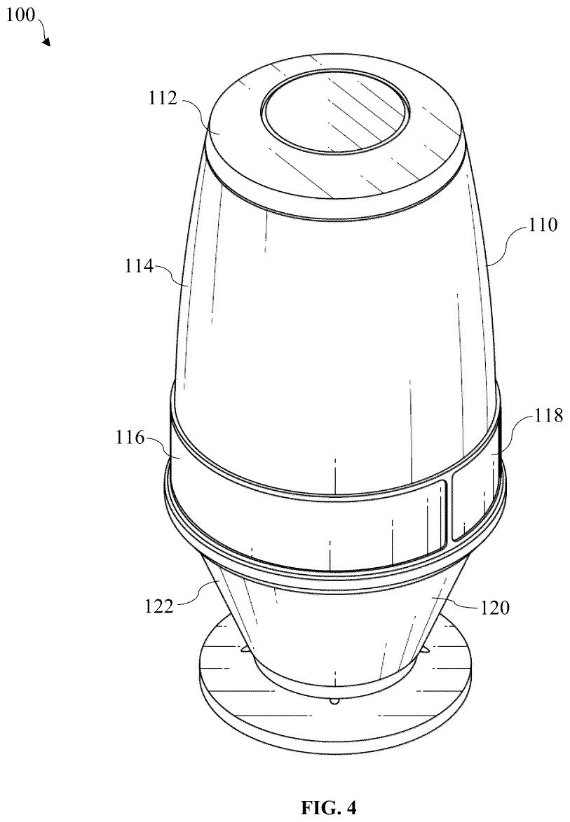

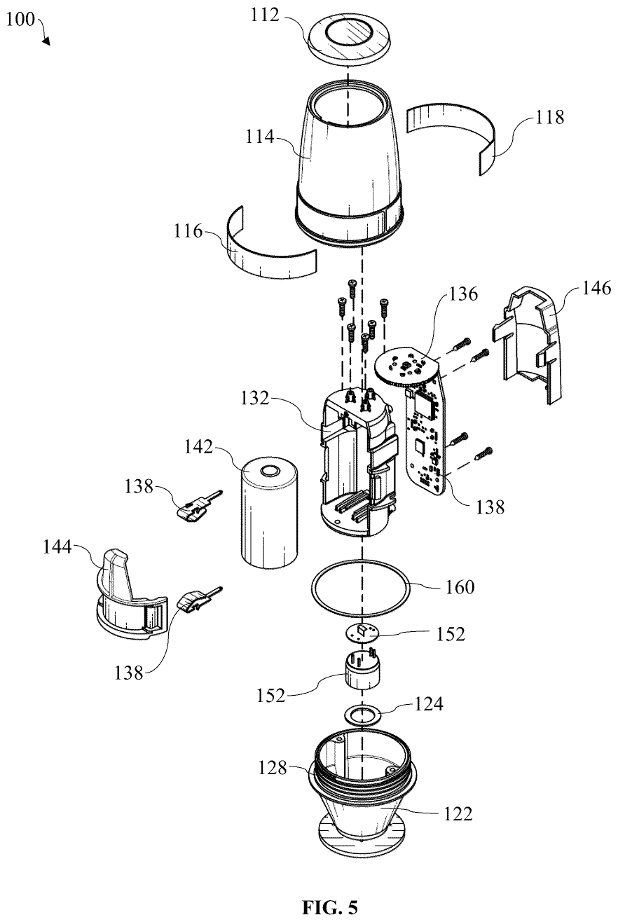

Referring to FIG. 5, the base housing 122 may also include base threads 128 to attach the housing assembly 110 thereon. A seal 160 such as an O-ring may be provided between the housing assembly 110 and the base assembly 120. FIG. 4 illustrates the purge gas monitor 100 when assembled according to some embodiments.

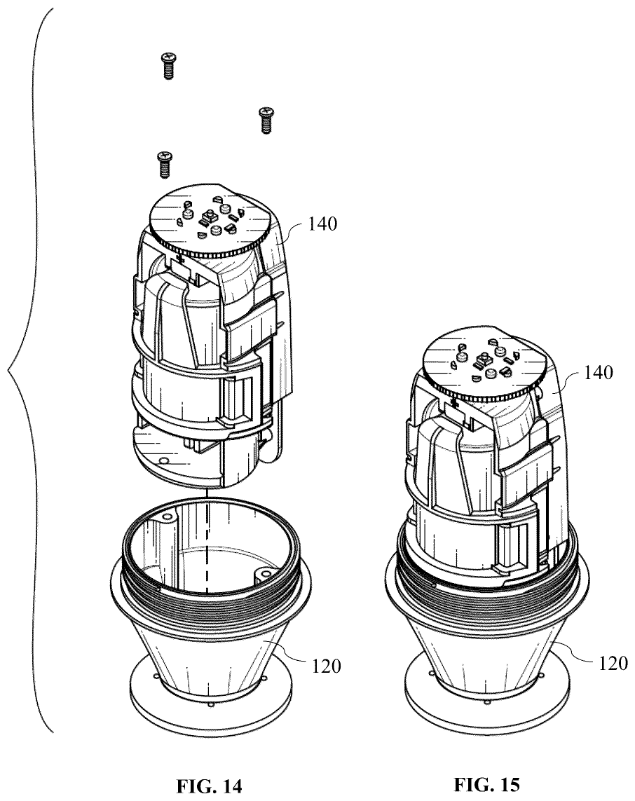

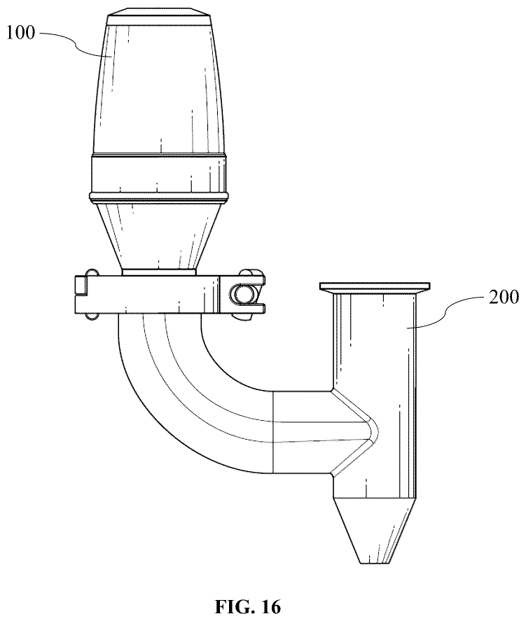

FIGS. 16-19 illustrate a pipe 200 that may be utilized in conjunction with the purge gas monitor 100 in the brewing system 10. The pipe 200 may include a first section 210 and a second section 220. The first section 210 may include a first funnel portion 212 proximal to a top end of the first section 210, a second funnel portion 214 proximal to a bottom end of the first section 210, and a straight portion 216 in between the first funnel portion 212 and the second funnel portion 214.

The first funnel portion 212 may include a first diameter proximal to a top end and a second diameter proximal to a bottom end. The top end of the first funnel portion 212 may be coupled to the outlet pipe 12 of the brewing system 10. The first diameter may be different than the second diameter. In some embodiments, the first diameter may be larger than the second diameter.

The straight portion 216 may have a generally uniform diameter throughout its length except for a location where the second section 220 intersects with the straight portion 216. In some embodiments, the uniform diameter of the straight portion 216 may be the second diameter.

The second funnel portion 214 may be frustoconical in shape. A top end of the second funnel portion 216 may be the second diameter, and a bottom end of the second funnel portion 216 may be a third diameter which may be smaller than the second diameter. The bottom end of the second funnel portion 214 may serve as the outlet 14 of the brewing system 10.

The second section 220 may include a third funnel portion 222 and a curved portion 224. The third funnel portion 222 may include a fourth diameter proximal to a top end and a fifth diameter proximal to a bottom end. The top end of the third funnel portion 222 may be coupled to the base assembly 120 of the purge gas monitor 100. The fourth diameter may be similar or the same as the first diameter. The fifth diameter may be similar or the same as the second diameter.

The curved portion 224 of the second section 220 be substantially parallel to the straight portion 216 proximal to the third funnel portion 222 and become substantially perpendicular to the straight portion 216 and intersects with the straight portion 216, thus resulting in the pipe 200 having a T-shape or a y-shape. Although described as the curved portion 224, it is to be understood that the curved portion 224 can be curved or have a substantially right angle. In further embodiments, the curved portion 224 may be substantially straight and slanted from the third funnel section 222 toward the straight portion 216.

As can be appreciated, through the pipe 200, a gas exiting the outlet pipe 12 may be in fluid communication between the first funnel portion 212, the second funnel portion 214, and the third funnel portion 222. Moreover, when the third diameter at the bottom end of the second funnel portion 216 is smaller than the second diameter, the exiting gas may be compressed and/or pressurized, ensuring the gas existing through the second funnel portion 214 would not reenter the pipe 200.

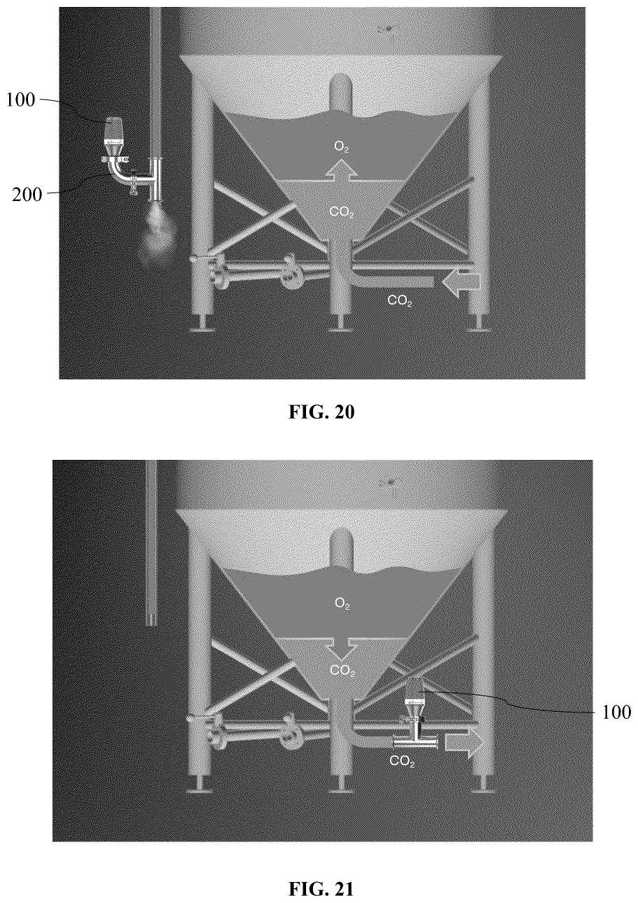

FIG. 20 illustrates an example configuration of the purge gas monitor 100 and the pipe 200 with respect to a brewing tank for monitoring an oxygen purge. As shown in FIG. 20, the purge gas monitor 100 and the pipe 200 may be installed proximal to an outlet of a purge gas, which is oxygen in this example. The purge gas monitor 100 can be used to monitor oxygen concentration as described above. Moreover, in the example illustrated in FIG. 20, the infill gas may be carbon dioxide. As explained in FIG. 1, data from the purge gas monitor 100 may be used by a controller for controlling a flow of the infill gas.

FIG. 21 illustrates an example configuration of the purge gas monitor 100 with respect to a brew tank for monitoring a carbon dioxide purge. In this example, an outlet pipe may be sealed or otherwise enclosed, forcing gas within the brew tank to compress and/or pressured. There, the purge gas monitor 100 may be provided on an inlet pipe. Instead of measuring and estimating when oxygen concentration may reach an acceptably low level, in this configuration, the purge gas monitor 100 may be configured to measure when oxygen concentration reaches an acceptably high level, which may signify that sufficient carbon dioxide has exited the brew tank.

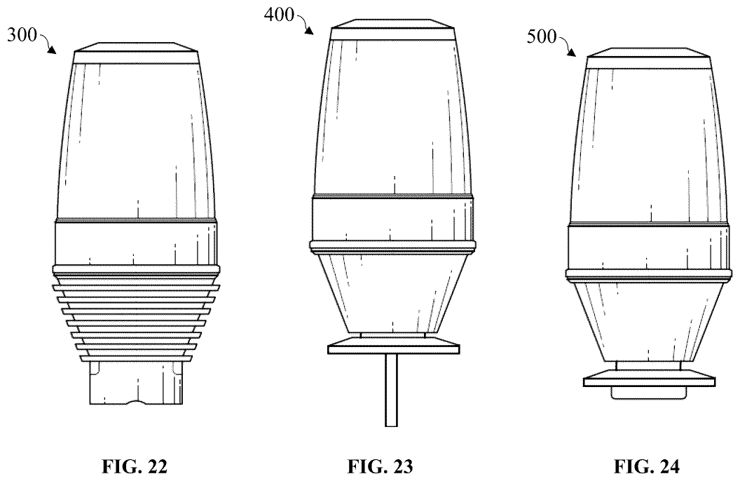

FIGS. 22-27 illustrate addition embodiments of various monitors that may be constructed using similar principles described above from constructing the purge gas monitor 100. Specifically, by swapping out the sensor 154 for other types of sensors, various monitors can be created.

FIG. 22 illustrates a chill monitor 300, where an internal sensor may be a thermometer suitable for operation in lower temperature ranges such as between about 0 degree Celsius to about 50 degree Celsius.

FIG. 23 illustrates a temperature monitor 400, where an internal sensor may be a thermometer suitable for operation in higher temperature ranges such as between about 50 degree Celsius to about 100 degree Celsius.

FIG. 24 illustrates a rinse monitor 500, where an internal sensor may be a liquid sensor suitable for monitoring liquid quality during rinsing.

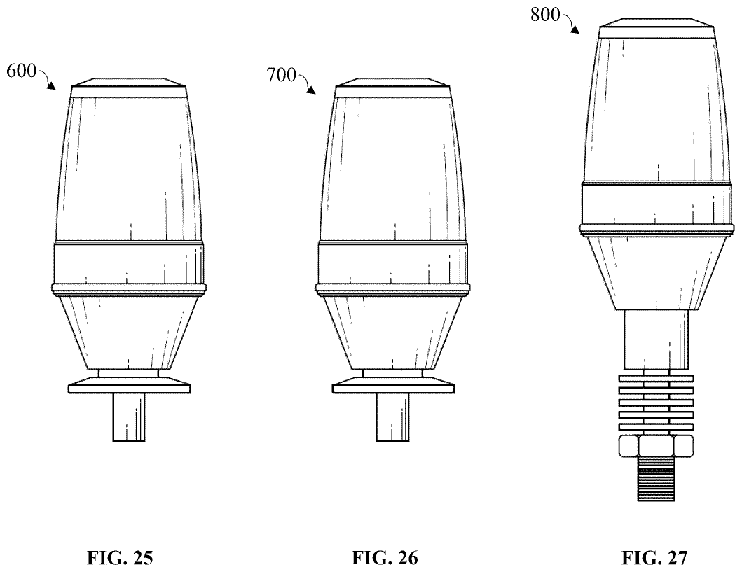

FIG. 25 illustrates a pH monitor 600, where an internal sensor may be an acidity sensor suitable for monitoring acidity levels of a liquid.

FIG. 26 illustrates a dissolved oxygen (DO) monitor 700, where an internal sensor may be a sensor suitable for monitoring dissolved oxygen (ppb level) of a liquid.

FIG. 27 illustrates a steam monitor 800, where an internal sensor may be a steam sensor suitable for monitoring a temperature and/or a content of a vaper.

Of course, may other types of monitors may also be constructed using the principles disclosed herein and are within the scope of this disclosure.

Specific embodiments of a purge gas monitor according to this disclosure have been described for the purpose of illustrating the manner in which the invention can be made and used. It should be understood that the implementation of other variations and modifications of this invention and its different aspects will be apparent to one skilled in the art, and that this invention is not limited by the specific embodiments described. Features described in one embodiment can be implemented in other embodiments. The subject disclosure is understood to encompass this disclosure and any and all modifications, variations, or equivalents that fall within the spirit and scope of the basic underlying principles disclosed and claimed herein.

What is claimed is:

1. A purge gas monitor comprising:
a housing assembly coupled to a base assembly, and
a sensor assembly provided in the base assembly,
wherein the sensor assembly includes an oxygen sensor.

2. The purge gas monitor of claim 1, wherein the housing assembly further comprises a lens coupled to a top end of a housing, wherein a bottom end of the housing is configured to be coupled to the base assembly.

3. The purge gas monitor of claim 1, wherein sensor assembly further comprises a sensor printed circuit board (PCB) coupled to the oxygen sensor.

4. The purge gas monitor of claim 3, wherein the sensor assembly is coupled to base assembly through a gasket provided between a bottom surface of the oxygen sensor and the base assembly.

5. The purge gas monitor of claim 4 further comprising a vent provided between the bottom surface of the oxygen sensor and the gasket.

6. The purge gas monitor of claim 1 further comprising a battery assembly, wherein the battery assembly is provided above the sensor assembly and is enclosed by the housing assembly when the purge gas monitor is assembled.

7. The purge gas monitor of claim 6, wherein the battery assembly further comprises a carrier for receiving a batter therein;
a first printed circuit board (PCB) provided on a first exterior surface of the carrier; and
a second PCB provided on a second exterior surface of the carrier.

8. The purge gas monitor of claim 7 further comprising:
a shroud attachable to the carrier, wherein the shroud encloses the first PCB when the shroud is attached to the carrier; and
a battery retainer attachable to the carrier, wherein the battery retainer retains the battery within the carrier when the battery retainer is attached to the carrier.

9. The purge gas monitor of claim 8, wherein the shroud and the battery retainer are provided on opposite sides of the carrier.

10. The purge monitor of claim 1, wherein a seal is provided in between the housing assembly and the base assembly.

11. A brewing system comprising:
a brew tank;
a purge gas monitor coupled to a pipe connected to the brew tank; and
a controller electronically coupled to the purge gas monitor,
wherein the controller is configured to:
receive a plurality of oxygen concentration measurements from the purge gas monitor over a period of time;
fit the plurality of oxygen concentration measurements onto a curve; and
extrapolate the curve to estimate a time when oxygen concentration will reach a threshold.

12. The brewing system of claim 11 further comprising:
a valve coupled to an actuator,
wherein the controller is further configured to:
in response to reaching the estimated time, control the actuator to actuate the valve.

13. The brewing system of claim 12, wherein the valve controls an infill gas flow into the brew tank.

14. The brewing system of claim 11, wherein the pipe that the purge gas monitor coupled to is an outlet pipe from the brew tank.

15. The brewing system of claim 11, wherein the pipe that the purge gas monitor coupled to is an inlet pipe to the brew tank.

16. The brewing system of claim 11, wherein the pipe further comprises a first section coupled to a second section, wherein the purge gas monitor is coupled to the second section of the pipe, and wherein a first end of the first section of the pipe is coupled to an outlet pipe from the brew tank and a second end of the first section serves as an outlet of a gas from the brew tank.

17. The brewing system of claim 16, wherein the first section of the pipe further comprises a straight portion coupled to a first funnel portion, wherein the straight portion comprises a generally uniform first diameter through a length of the straight portion.

18. The brewing system of claim 16, wherein the first funnel portion includes a first end having the first diameter and a second end having a second diameter different from the first diameter.

19. The brewing system of claim 16, wherein the second section of the pipe intersects the first section of the pipe at the straight portion.

20. The brewing system of claim 19, wherein a first end of the second section where the purge gas monitor is coupled to is substantially parallel to the straight portion and a second end of the second section where the second section intersects the first section is substantially perpendicular.
